(19) United States
(12) Patent Application Publication
(10) Pub. No.: US 2022/0249112 A1
(43) Pub. Date: Aug. 11, 2022

(54) ANGULAR POSITIONING SYSTEM FOR ROTARY SURGICAL INSTRUMENT

(71) Applicant: GYRUS ACMI, INC.D/B/A OLYMPUS SURGICAL TECHNOLOGIES AMERICA, Westborough, MA (US)

(72) Inventors: Joey Magno, Dudley, MA (US); Rodney Loyd, Arlington, TN (US)

(21) Appl. No.: 17/650,299

(22) Filed: Feb. 8, 2022

Related U.S. Application Data
(60) Provisional application No. 63/147,480, filed on Feb. 9, 2021.

(51) Int. Cl. A61B 17/32; A61B 90/00

(52) U.S. Cl. CPC A61B 17/32002; A61B 2017/00128; A61B 90/08

(57) ABSTRACT

A powered surgical instrument can include a cutting assembly including an outer tubular member defining an outer cutting window, an inner tubular member arranged to be rotatable concentrically within the outer tubular member and defining an inner cutting window, and a control system. The control system can include a controller, an angular position sensor to provide to the controller, with respect to at least one of the inner and outer tubular members, an indication of angular orientation therebetween to allow the controller to control the angular orientation of the inner cutting window relative to the outer cutting window, without requiring user intervention, such that the inner and outer cutting windows are aligned when relative rotation between the inner and outer tubular members is stopped or paused.

### CROSS-REFERENCE TO RELATED APPLICATION

This application claims the benefit of priority to U.S. Provisional Patent Application Ser. No. 63/147,480, filed Feb. 9, 2021, the contents of which are incorporated herein by reference in their entirety.

### TECHNICAL FIELD

The present application pertains generally, but not by way of limitation, to devices and methods for performing surgical procedures with rotary cutting instruments.

### BACKGROUND

During some surgical procedures, such as a keyhole, or minimally invasive, operation, a rotary surgical cutting instrument can be used to cut, resect, debride, or otherwise remove tissue and/or bone from inside a patient's body through a small access incision, such as in laparoscopic, arthroscopic, endoscopic, or in various ear, nose, and throat (“ENT”) operations. Such rotary cutting instruments often include a cutting assembly having an inner tubular member concentrically located within an outer tubular member. The inner and outer tubular members each can define a cutting window extending through an annular surface of a distal portion. The inner cutting window can be rotationally driven by, for example, an electric motor and the outer cutting window can generally remain stationary. In this regard, fragments of tissue or bone can be resected each time the rotating inner cutting window rotates past the stationary outer cutting window.

### SUMMARY/OVERVIEW

Rotary cutting instruments can be used to visualize, resect, and extract tissue or bone from various regions of a patient's body during keyhole surgical procedures. Such cutting instruments can include a cutting assembly, including inner and outer tubular members, extending distally from a housing, such as a handle or a handpiece. The cutting assembly can be inserting into an access incision or a body cavity to reach a target surgical site. A proximal end of the inner tubular member can engage an electric motor within the housing to provide rotational drive to the inner tubular member. The inner cutting window is typically oscillated within the outer tubular member using an open-loop control system, such a time based system, with the inner tubular member being rotationally driven for a specified amount of time in a first, or forward, direction and then subsequently being rotationally driven in a second, and opposite, direction for a specified amount of time.

A rotary cutting instrument can be used with an external vacuum source to provide suction through the inner cutting window, to evacuate surgical debris from the surgical site. The inner tubular member can be hollow to form a portion of a suction passage that can extend through the inner tubular member and generally through the housing to the external vacuum source. The use of suction can both improve visibility at the surgical site, and increase the cutting efficiency of a cutting instrument, by drawing in tissue for resection and removing surgical debris which can reduce visibility or inhibit movement of the inner cutting window.

A rotary cutting instrument can also be used with an external fluid source to provide irrigation fluid to a surgical site. Irrigation fluid can also be supplied to a surgical site by the cutting instrument. For example, in ENT procedures, a surgical site may lack a fluid medium, and thus it is can be desirable to introduce irrigation fluid to reduce the possibility of clogging the inner tubular member with surgical debris. The irrigation fluid can generally enter a surgical site through a gap defined between the inner tubular member and outer tubular member and exit the surgical site by being sucked through the inner cutting window of the inner tubular member.

The present inventors have recognized, among other things, that an oscillating rotary cutting instrument can operate most efficiently if the inner and outer cutting windows are aligned each time the inner tubular member reverses rotation direction during an oscillation cycle, as suction flow from the vacuum source, and correspondingly, the rate of removal of surgical debris and/or irrigation fluid evacuation from the surgical site is at its highest when the inner and outer cutting windows are aligned. Existing open-loop control systems, such as time-based systems, do not monitor the position of the inner cutting window relative to the outer cutting window during an oscillation cycle.

Thus, each time the inner cutting window reverse rotation direction during an oscillation cycle, it comes to a stop in an uncontrolled angular orientation relative to the outer cutting window. Ineffective alignment of the inner cutting window relative to the outer cutting window can result in debris clogging the inner tubular member to a point where it is necessary to frequently stop oscillation of the inner tubular member to allow time for effective aspiration of fluid and debris. This can significantly prolong the length of a surgical procedure. Additionally, tissue can be pinched or torn between the inner and outer cutting windows, rather than being effectively cut, if the inner cutting window comes to a stop in a position substantially offset from the outer cutting window.

Moreover, existing methods of aligning the inner cutting window with the outer cutting window are not suitable for use during an oscillation cycle. As oscillating cutting instruments rapidly change the rotational direction of the inner tubular member, often rotating in a single direction for a few hundred milliseconds, it is not possible for a user to manually intervene to align the inner cutting window with the outer cutting window each time the inner tubular member switches directions, for example, by pressing an electronic indexing feature, or by manually indexing the inner cutting window into alignment with the outer cutting window once rotation is paused.

This disclosure can help to address these issues, among others, such as by providing an angular positioning system capable of implementing automatic alignment of the inner and outer cutting windows of a rotary surgical cutting instrument each time relative rotation between the inner and outer tubular members is paused during an oscillation cycle to improve the cutting efficiency of the cutting instrument. The angular positioning system can also allow a physician to configure a dwell time of the inner tubular member relative to the outer tubular member to improve the adaptability of a rotary cutting instrument to respond to intra-procedural conditions.

Additionally, it can be desirable to provide a fluid medium at the surgical site by maintaining fluid pressure at or around the surgical site, such as in an ENT procedure. In such a procedure, the angular positioning system can allow a user to stop the inner cutting window in an angular position substantially opposite relative the outer cutting window to prevent a loss of fluid pressure during tissue resection. Preventing aspiration through the inner tubular member can also allow the outer tubular member to be used as probe, to manipulate anatomy, within the access incision without drawing in tissue to reduce the need to withdraw the cutting assembly for substitution with a separate surgical tool, which can lengthen the procedure. Thus, the angular positioning system can improve the cutting efficiency of a rotary cutting instrument, increase intra-procedural visibility for a physician operating a rotary cutting instrument, and improve the adaptability of a rotary cutting instrument to response to particular intra-procedural conditions to reduce the length of a keyhole surgical procedure.

The above overview is intended to provide an overview of subject matter of the present patent application. It is not intended to provide an exclusive or exhaustive explanation of the invention. The description below is included to provide further information about the present patent application. While the following examples are discussed with a focus toward cutting instruments configured for ENT procedures, the angular positioning system can also be used in various other rotary cutting instruments configured for other procedures, such as in arthroscopic or laparoscopic cutting instruments.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a cross-section of an example of a surgical cutting instrument including an angular positioning system.

FIG. 2A illustrates a cross-section of an example of a cutting assembly of a surgical cutting instrument.

FIG. 2B illustrates a perspective view of an example of a cutting assembly of a surgical cutting instrument.

FIG. 3 illustrates an example of a motor oscillation cycle of a surgical cutting instrument.

FIG. 4A illustrates an example of a portion of a motor oscillation cycle of a surgical cutting instrument.

FIG. 4B illustrates an example of a portion of a motor oscillation cycle of a surgical cutting instrument.

FIG. 4C illustrates an example of a portion of a motor oscillation cycle of a surgical cutting instrument.

FIG. 5 illustrates a schematic view of an example of an angular positioning system for a surgical cutting instrument.

FIG. 6 illustrates an example of a method of controlling a surgical cutting instrument.

### DETAILED DESCRIPTION

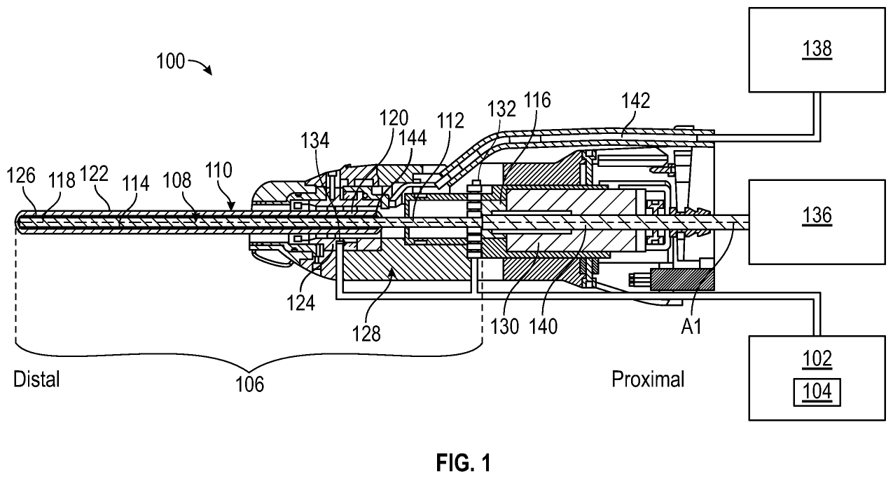

FIG. 1 illustrates a cross-section of an example of a surgical cutting instrument 100 including an angular positioning system 102. FIG. 1 includes a dashed line corresponding to a central longitudinal axis A1 defined by the surgical cutting instrument, and orientation indicators “Proximal” and “Distal”. The angular positioning system 102 can be configured to control several components and operations of a surgical cutting instrument 100. The angular positioning system 102 can include a controller 104. The controller 104 can include processing circuitry or hardware. The controller 104 can be user-programmable to selectively configure several parameters or operations of the angular positioning system 102.

The surgical cutting instrument 100 can include a cutting assembly 106. The cutting assembly 106 can include an inner tubular member 108 and an outer tubular member 110. The outer tubular member 110 can be configured to concentrically receive the inner tubular member 108. For example, the inner tubular member 108 can be inserted into the outer tubular member 110, such that the outer tubular member 110 can radially or otherwise laterally encompass the inner tubular member 108. The inner tubular member 108 can include a proximal portion 112 and a distal portion 114. The cutting assembly 106 can include an inner hub 116. The proximal portion 112 can be coupled to the inner hub 116. The distal portion 114 can define an inner cutting window 118 extending through an annular, or side surface, of the inner tubular member 108.

The outer tubular member 110 can include a proximal portion 120 and a distal portion 122. The cutting assembly 106 can include an outer hub 124. The proximal portion 120 can be coupled to, and extend axially through, the outer hub 124. The distal portion 122 can define an outer cutting window 126 extending through an annular, or side surface of the outer tubular member 110. The cutting assembly 106 can be a disposable, or single use, cutting assembly. For example, the cutting assembly 106 can be decoupled from the inner hub 116 and discarded after a surgical procedure. The cutting assembly 106 can also be configured to be reprocessed, autoclaved, or otherwise sterilized, along with other components of the surgical cutting instrument 100, for reuse in a subsequent procedure. The surgical cutting instrument 100 can include a housing 128. The housing 128 can generally be a handpiece or handle of the surgical cutting instrument 100. The distal portion 114 of the inner tubular member 108, and the distal portion 122 of the outer tubular member 110 can extend axially and distally from the housing 128, along the longitudinal axis A1. The housing 128 can be configured to encompass various components of the surgical cutting instrument 100. For example, the housing 128 can encompass the proximal portions 112 and 120 of the inner tubular member 108 and outer tubular member 110, respectively.

The housing 128 can encompass and position a motor 130 with respect to the inner hub 116. The motor 130 can be a cannulated motor. The motor 130 can, based on an output of the controller 104, rotate the inner hub 116 to rotate the inner tubular member 108 in a forward and in a reverse direction. For example, the controller 104 can output a signal to the motor 130 to oscillate the inner tubular member 108 within the outer tubular member 110. The angular positioning system 102 can include at least a first angular position sensor 132. The angular positioning system 102 can also include a second angular position sensor 134. The first angular position sensor 132 and the second angular position sensor 134 can be located within the housing 128.

The first angular position sensor 132 can be configured to monitor an angular position of the inner cutting window 118. The second angular position sensor 134 can be configured to monitor an angular position of the outer cutting window 126, in examples where the outer tubular member 110 is rotatable relative to the inner tubular member 108. The first angular position sensor 132 and the second angular position sensor 134 can be in signal communication with the controller 104, such as to provide an indication of angular orientation therebetween. The controller 104 can use processing circuitry to determine an angular position of the inner cutting window 118 relative to the outer cutting window 126, such as to control alignment of the inner cutting window 118 relative to the outer cutting window 126. The angular positioning system 102 can thereby align the inner cutting window 118 with the outer cutting window 126, when relative rotation between the inner tubular member 108 and the outer tubular member 110 is paused, such as when the inner tubular member 108 comes to a stop in order to reverse rotational direction during an oscillation cycle. When the inner cutting window 118 is aligned with the outer cutting window 126, the surgical cutting instrument 100 can be in an open position.

In some examples, such as during an ENT procedure, the surgical cutting instrument 100 can also include a vacuum source 136 and a fluid source 138. The vacuum source 136 and the fluid source 138 can be located externally to the housing 128. The vacuum source 136 can engage a suction passage 140. The suction passage 140 can extend axially and distally within the housing 128, along the longitudinal axis A1. The suction passage 140 can be partially defined by the inner tubular member 108. For example, the inner tubular member 108 can be hollow to comprise a portion of the suction passage 140, to the extent that the inner tubular member 108 extends within the housing 128. The suction passage 140 can extend distally through the motor 130, the inner hub 116, the outer hub 124, and through the inner tubular member 108, to provide suction through the inner cutting window 118. The fluid source 138 can engage a fluid passage 142 extending distally through the housing 128. The fluid passage 142 can be partially defined by a gap 144 maintained between the inner tubular member 108 and the outer tubular member 110. The fluid source 138 can thereby provide irrigation fluid to a surgical site.

The angular positioning system 102 can provide several benefits to a patient and to a physician. For example, the controller 104 can increase the cutting efficiency of a rotary surgical cutting instrument by improving the rate of aspiration of surgical debris and irrigation fluid from a surgical site, as well as more effectively drawing in tissue for resection, during an oscillation cycle. This can reduce the length of a surgical procedure. For example, a user can, via the controller 104, activate the motor 130 to oscillate the inner tubular member 108 within the outer tubular member 110, to, for example, resect tissue within the body of a patient. The controller 104 can use processing circuitry to process signals received from the first angular position sensor 132 and/or the second angular position sensor 134 to modify an output to the motor 130, to concurrently stop the inner tubular member 108 and bring the inner cutting window 118 into alignment with the outer cutting window 126, such as to bring the surgical cutting instrument 100 into the open position, each time the inner tubular member 108 reverses its rotational direction.

The angular positioning system 102 can also improve the adaptability of a rotary cutting instrument to respond to intra-procedural conditions. For example, the controller 104 can allow a physician to configure a dwell time of the inner tubular member 108 relative to the outer tubular member 110, to increase or decrease suction flow through the inner cutting window 118 during an oscillation cycle. Further, some rotary cutting instruments, such as those configured for ENT procedures, allow a physician to manually rotate an outer tubular member relative to the inner tubular member, to improve access to a target surgical site by selecting a desirable angular position of the outer cutting window.

The angular positioning system 102 can concurrently monitor the angular positions of both the inner tubular member 108 and the outer tubular member 110 to automatically bring the surgical cutting instrument 100 into the open position, such as by bringing the inner cutting window 118 into alignment with the outer cutting window 126 each time the inner tubular member 108 reverses direction, to allow a physician to change the angular orientation of the outer tubular member 110 pre-procedurally or intra-procedurally while maintaining the benefit of automatic alignment of the inner cutting window 118 and the outer cutting window 126.

The angular positioning system 102 can further reduce the length of the surgical procedure by maintaining fluid pressure at or near a surgical site during an oscillation cycle. For example, the controller 104 can allow a physician to selectively configure the controller 104 to implement automatic misalignment of the inner cutting window 118 and the outer cutting window 126 each time the inner tubular member 108 reverses rotational direction, to reduce irrigation fluid loss through the inner cutting window 118 and avoid depressurization of the surgical site.

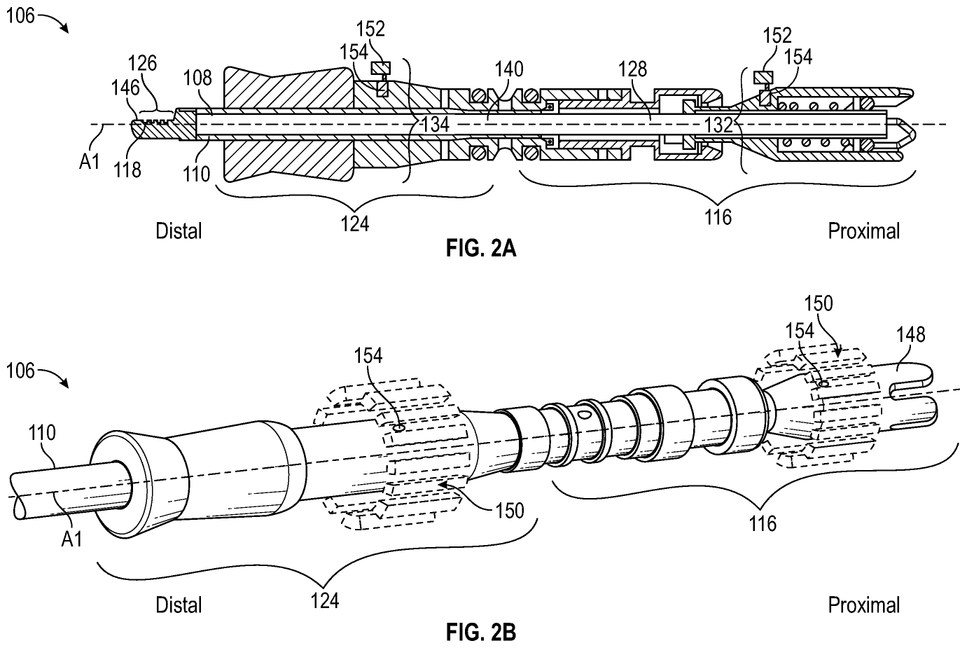

FIG. 2A illustrates a cross-section of an example of a cutting assembly 106 of a surgical cutting instrument. FIG. 2B illustrates a perspective view of an example of a cutting assembly 106 of a surgical cutting instrument. FIGS. 2A-2B include a dashed line corresponding to a central longitudinal axis A1, and orientation indicators “Proximal” and “Distal”. FIGS. 2A-2B are discussed below concurrently.

As illustrated in FIG. 2A, the inner cutting window 118 can define a cutting blade 146. For example, the inner cutting window 118 can define a single sharpened edge extending generally parallel to the longitudinal axis A1. The cutting blade 146 can also define two or more sharpened edges, such as a plurality of cutting teeth extending at various angles relative to the longitudinal axis A1. The inner hub 116 can define a coupler 148. The coupler 148 can generally be a proximal end of the inner hub 116. The coupler 148 can be configured to detachably couple the inner hub 116 to a shaft of a motor, such as the motor 130 shown in FIG. 1, to provide rotational drive to the inner tubular member 108.

The angular positioning system 102, such as the angular positioning system 102 shown in FIG. 1, can include the first angular position sensor 132. Alternatively, the angular positioning system 102 can include the first angular position sensor 132 and the second angular position sensor 134. The outer tubular member 110 can be rotatable relative to the inner tubular member 108 and to the housing 128 shown in FIG. 1. For example, the outer tubular member can be manually rotated between 0 and 360 degrees relative to the housing 128. In such an example, the angular positioning system 102 can include both the first angular position sensor 132 and the second angular position sensor 134, to concurrently monitor the angular positions of both the inner cutting window 118 and the outer cutting window 126. The first angular position sensor 132 and the second angular position sensor can be in signal communication with a controller, such as the controller 104 shown in FIG. 1.

The first angular position sensor 132 and the second angular position sensor 134 can output signals to the controller 104. The signals can be an indication of, or otherwise correspond to, the angular positions of the inner tubular member 108 and the outer tubular member 110, respectively. The first angular position sensor 132 and the second angular position sensor 134 can be a variety of sensors configured to generate electrical signals to the controller 104. The first angular position sensor 132 and the second angular position sensor 134 can each include a reference feature 150, such as shown in FIG. 2B, and a sensing component 152, such as shown in FIG. 2A. The reference feature 150 of each of the first angular position sensor 132 and the second angular position sensor 134 can extend around a circumference of the inner hub 116 and the outer hub 124. The reference feature 150 can include a marker 154 located on a point around the circumference of the reference feature 150. The reference feature 150 can, for example, be a collar or a sleeve configured to receive and retain the marker 154.

The sensing component 152 can be fixedly located within the housing 128 of a surgical cutting instrument, such as the surgical cutting instrument 100 shown in FIG. 1. The sensing component 152 of each of the first angular position sensor 132 and the second angular position sensor 134 can be positioned with respect to the inner hub 116 and the outer hub 124, respectively. The sensing component 152 can output an electrical signal, such as a digital or analog signal, each time the marker 154 of the reference feature 150 aligns with the sensing component 152 during rotation, such as to provide an indication of an angular position of the inner tubular member 108 or the outer tubular member 110, to the controller 104. Therefore, in at least one example, the meaning of “indication” can be an electrical signal output to the controller 104 from one or more angular position sensors, such as the first angular position sensor 132 or the second angular position sensor 134.

Each marker 154 can be circumferentially aligned with the inner cutting window 118 and the outer cutting window 126, such that the signals of the first angular position sensor 132 and the second angular position sensor 134 accurately represent the angular positions of the inner cutting window 118 and the outer cutting window 126. The controller 104 can process the signals of the first angular position sensor 132 and the second angular position sensor 134 to determine the angular position of the inner cutting window 118 relative to the outer cutting window 126. The controller 104 can control alignment of the inner cutting window 118 and the outer cutting window 126. For example, the controller 104 can modify a motor signal to the motor 130 during an oscillation cycle, to pause rotation between the inner tubular member 108 and the outer tubular member 110, in preparation of reversing the rotational direction of the inner tubular member 108, to align the inner cutting window 118 with the outer cutting window 126.

When the controller 104 senses the inner tubular member 108 has rotated a specified number of rotations, the controller 104 can modify the motor signal to cause the motor 130 to decelerate until the inner cutting window 118 is stopped and in alignment with the outer cutting window 126, such as to bring the surgical cutting instrument 100 into the open position. The angular positioning system 102 can also be configured to align the inner cutting window 118 with the outer cutting window 126 with varying degrees of tolerance. For example, depending on the accuracy of the first angular position sensor 132 and the second angular position sensor 134, alignment of the inner cutting window 118 relative to the outer cutting window 126 can deviate, or be offset by, about 0-5, 5-10, 15-20, or 2-20 degrees. Therefore, the meaning of “alignment” can include a partially, substantially or nearly aligned state depending on the accuracy.

Thus, the angular positioning system 102 can form a closed-loop control system capable of automatically aligning the inner cutting window 118 and the outer cutting window 126 during oscillation of the inner tubular member 108, without requiring a user intervention, such as engaging (or disengaging) stop control on the housing 128, controller 104, or an external foot pedal, to stop rotation of the inner tubular member relative to the outer tubular member. The controller 104 can also be user configurable. For example, a physician can specify, via a user-input, the length of time the inner tubular member 108 is to remain stationary relative to the outer tubular member 110 relative rotation between the inner tubular member 108 and the outer tubular member 110 is paused. A physician can also specify, via a user-input, whether the controller 104 is to stop the inner cutting window 118 in alignment or out of alignment with the outer cutting window 126. For example, the controller 104 can be configured to stop the inner cutting window 118 in a position about 150-160, 160-170, or 150-180 degrees offset relative to the outer cutting window 126.

In one or more examples, the first angular position sensor 132 and the second angular position sensor 134 can be hall-effect sensors. For example, the marker 154 of the reference feature 150 can be a magnet, and the sensing component 152 can be a hall plate. A digital or analog signal, such as a pulse or voltage peak, can be output to the controller 104 when the marker 154 is circumferentially aligned with the sensing component 152. In one or more examples, the first angular position sensor 132 and/or the second angular position sensor 134 can be SL353 MicroPower Omnipolar Digital Hall Effect ICs sensors.

In one or more examples, the first angular position sensor 132 and the second angular position sensor 134 can be optical sensors. For example, the marker 154 of the reference feature can be a generally dark colored marking, such as a dot or a bar, or a series of markings, located on a point or a series of points around the circumference of the reference feature 150. In such examples, the reference feature 150 can generally be reflective to help the optical sensor identify the marker 154 within the reference feature. The sensing component 152 can be, for example, a photodiode or a phototransistor. The sensing component 152 can also include a light emitting diode (“LED”) to illuminate the reference feature 150 at a wavelength of sensitivity of the sensing component 152. A digital or analog signal, such as a pulse or voltage peak, can be output to the controller 104 when the marker 154 becomes circumferentially aligned with the sensing component 152, momentarily blocking the reflection from the reference feature 150. In one or more examples, the first angular position sensor 132 and/or the second angular position sensor 134 can be VCNT2020 Reflective Optical Sensors from Vishay Intertechnology, or OPB9000 SMD reflective optical sensors from TT Electronic/Optek Technology.

In one or more examples, the first angular position sensor 132 can be an optical encoder incorporated into the motor 130. In such examples, the optical encoder can output a continuous digital signal to the controller 104 corresponding to the angular position of the inner cutting window 118 by monitoring the angular position of the motor 130, relative to a known reference point, such as the angular position of the outer cutting window 126. Thus, when the inner tubular member 108 is coupled to the motor 130, the optical encoder can detect alignment between the inner cutting window 118 and the outer cutting window 126.

In still further examples, the first angular position sensor 132 and the second angular position sensor 134 can each include, or otherwise comprise, for example, a mechanical sensing arrangement such as a spring-loaded electrical contact, or a capacitive or inductive sensing arrangement. In at least one example including a mechanical sensing arrangement, the meaning of “indication” can be a mechanical action or output such as physical engagement between one or more spring loaded mechanical contacts, or a physical engagement between a cam and a cam follower, from one or more angular position sensors, such as the first angular position sensor 132 or the second angular position sensor 134.

The angular positioning system 102 can also include various combinations of examples of the first angular position sensor 132 and the second angular position sensor 134 described above. For example, the first angular position sensor 132 can be an optical encoder, and the second angular position sensor 134 can be a hall-effect, or an optical, sensor.

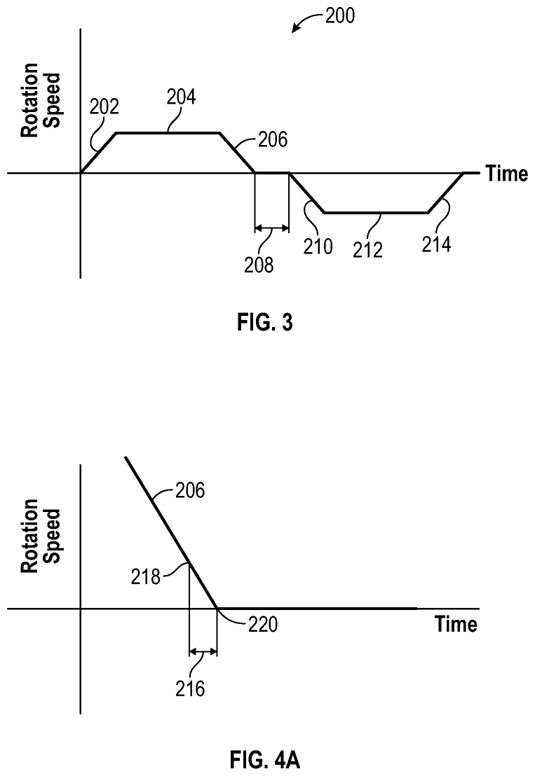

FIG. 3 illustrates an example of a motor oscillation cycle 200. FIG. 3 is discussed with reference to FIGS. 1-2B above. As illustrated in FIG. 3, the varying rotation speed of the inner tubular member 108 during an oscillation cycle can be graphically shown. The motor oscillation cycle 200 can begin with a first acceleration phase 202. For example, the motor 130, in response to receiving a motor signal from the controller 104, can begin to rotate the inner tubular member 108 in a first, or forward, direction. The motor 130 can continuously increase the rotation speed of the inner tubular member 108, until the controller 104 determines that the motor 130 has reached a specified maximum rotation speed. The maximum rotation speed can be selectively configured via the controller 104. For example, the maximum rotation speed can be about, but not limited to, 200-300, 360-420, or 120-480 rpm. In some examples, the acceleration rate of the motor can be selectively configured via the controller 104 to increase or decrease the length of the acceleration phase.

The motor oscillation cycle 200 can include a first rotation phase 204. During the first rotation phase, the inner tubular member 108 can be rotated a specified amount (e.g., time or distance), such as a specified whole or fractional number of rotations in a first, or forward, direction at a constant rotation speed. In some examples, the specified number of rotations can be selectively configured via the controller 104 to increase or decrease the length of the first rotation phase 204. For example, the inner tubular member 108 can complete about 4-6, 5-8, or 5-10 rotations during the first rotation phase 204, though other numbers of rotations can be specified. The motor oscillation cycle 200 can include a first deceleration phase 206. After the inner tubular member 108 rotates the specified number of rotations at 204, the controller 104 can stop or modify the motor signal to the motor 130, to begin to reduce the rotation speed of the motor 130 until the inner tubular member 108 ceases rotation relative to the outer tubular member 110, and the inner cutting window 118 is aligned with the outer cutting window 126, such as to bring the surgical cutting instrument 100 into the open position.

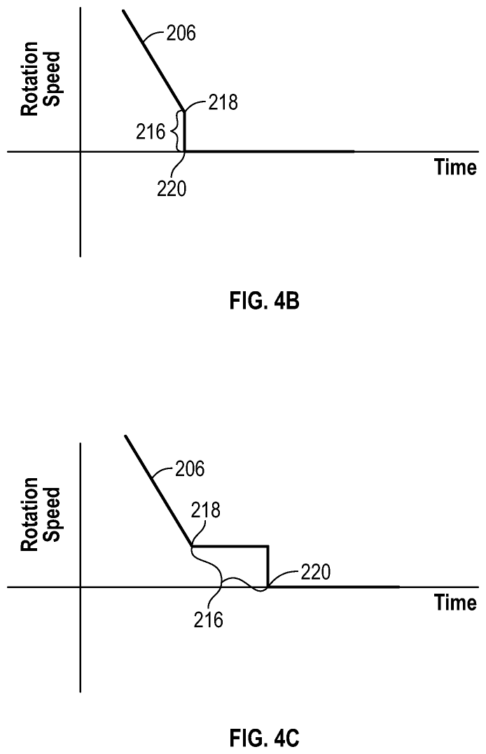

The controller 104 can process signals from the first angular position sensor 132, or concurrently process signals from both the first angular position sensor 132 and the second angular position sensor 134, to reduce the rotation speed of the motor 130 such that the inner cutting window 118 of the inner tubular member 108 comes to a rest in a position aligned with the outer cutting window 126 during a dwell time 208, such as discussed in FIGS. 4A-4C below. The motor oscillation cycle 200 can also include a second acceleration phase 210, a second rotation phase 212, and a second deceleration phase 214. The second acceleration phase 210, the second rotation phase 212, and the second deceleration phase 214 can be similar to the first acceleration phase 202, the first rotation phase 204, and the first deceleration phase 206, except that the controller 104 can rotate the inner tubular member 108 in a second, or reverse, direction.

FIGS. 4A-4C illustrate examples of portions of a motor oscillation cycle 200 of a surgical cutting instrument. FIGS. 4A-4C are discussed below concurrently. FIGS. 4A-4C can represent different examples of the first deceleration phase 206 or the second deceleration phase 214 of the motor oscillation cycle 200 as discussed above with reference to FIG. 3. For convenience and clarity, the following examples are discussed with regard to the first deceleration phase 206. As illustrated in FIGS. 4A-4C, the first deceleration phase 206 can include a window alignment phase 216. The window alignment phase 216 can generally be defined as a time period between when the controller 104 outputs a motor stop signal, after determining that the inner tubular member 108 has rotated, for example, the specified number of rotations in the first rotation phase 204, and the time the inner tubular member 108 comes to a stop, relative to the outer tubular member 110.

FIG. 4A illustrates a linear example of the first deceleration phase 206. The controller 104 can control alignment of the inner cutting window 118 and the outer cutting window 126 during the window alignment phase 216. For example, the window alignment phase 216 can begin at point 218, with the controller 104 determining, using signals from the first angular position sensor 132 and the second angular position sensor 134 indicating the angular orientation therebetween, that the inner cutting window 118 has rotated a specified number of complete rotations. The controller 104 can then output a motor stop signal to the motor 130. At point 220, rotation of the motor 130 and inner tubular member 108 relative to the outer tubular member 110 is stopped. As illustrated in FIG. 4A, based on the rotation speed of the motor 130, the controller 104 can decelerate the motor 130, and correspondingly, the inner tubular member 108 to continue decelerating at a constant, or linear, rate until the controller 104 receives a signal from the first angular position sensor 132 and the second angular position sensor 134 indicating that the inner cutting window 118 is aligned, or is nearly aligned, with the outer cutting window 126, at which point the controller 104 can stop the motor 130.

For example, the controller 104 can output a motor stop signal to significantly slow the rotation speed of the motor 130, such as by cutting power to the motor to allow friction inherent within the motor to slow rotation of the inner tubular member 108, and monitor the output of the first angular position sensor 132 and the second angular position sensor 134 and thus the angular orientation therebetween, until the controller 104 determines that the surgical cutting instrument 100 is in the open position, with the inner cutting window 118 aligned with the outer cutting window 126. As can be appreciated, the motor 130 may not consistently come to a stop in an angular position causing the inner cutting window 118 and the outer cutting window 126 to be aligned, if only a passive deceleration technique is implemented by the controller 104. As such, FIGS. 4B and 4C illustrate examples of how the controller 104 can control or otherwise ensure alignment between the inner cutting window 118 and the outer cutting window 126 if the controller 104 determines that the inner tubular member 108 will not come to a stop in an angular position circumferentially aligning the inner cutting window 118 with the outer cutting window 126.

For example, FIG. 4B illustrates how the controller 104 can implement active braking at point 218, during the first deceleration phase 206, to ensure the motor 130 comes to stop in an angular position aligning the inner cutting window 118 and the outer cutting window 126, at point 220. In some examples, after the controller 104 has output a motor stop signal, the controller 104 can be configured to additionally output an active braking signal to abruptly stop rotation of the motor 130. For example, if the rotation speed of the motor 130 is below a specified threshold speed when the controller 104 determines, based on the signals from the first angular position sensor 132 and the second angular position sensor 134 indicating that the surgical cutting instrument 100 is in the open position with the inner cutting window 118 and the outer cutting window 126 aligned, the controller 104 can output an active braking signal to immediately or otherwise abruptly stop rotation of the motor 130.

FIG. 4C illustrates how the controller 104 can implement an additional rotation of the inner tubular member 108, during the first deceleration phase 206, to ensure the motor 130 comes to stop in an angular position aligning the inner cutting window 118 and the outer cutting window 126, at point 220. In some examples, after the controller 104 has output a motor stop signal, the controller 104 can be configured to additionally output a modified motor signal to cause the motor to rotate at a reduced rate of speed. For example, if the rotation speed of the motor 130 is at or above a specified threshold speed when the controller 104 determines, based on the signals from the first angular position sensor 132 and the second angular position sensor 134, that the surgical cutting instrument 100 is in the open position with the inner cutting window 118 and the outer cutting window 126 aligned, the controller 104 can output a modified motor signal to cause the motor 130 to continue rotating the inner tubular member at a reduced rate of speed until the controller 104 receives signals from the first angular position sensor 132 and the second angular position sensor 134 indicating alignment the inner cutting window 118 and the outer cutting window 126 are circumferentially aligned.

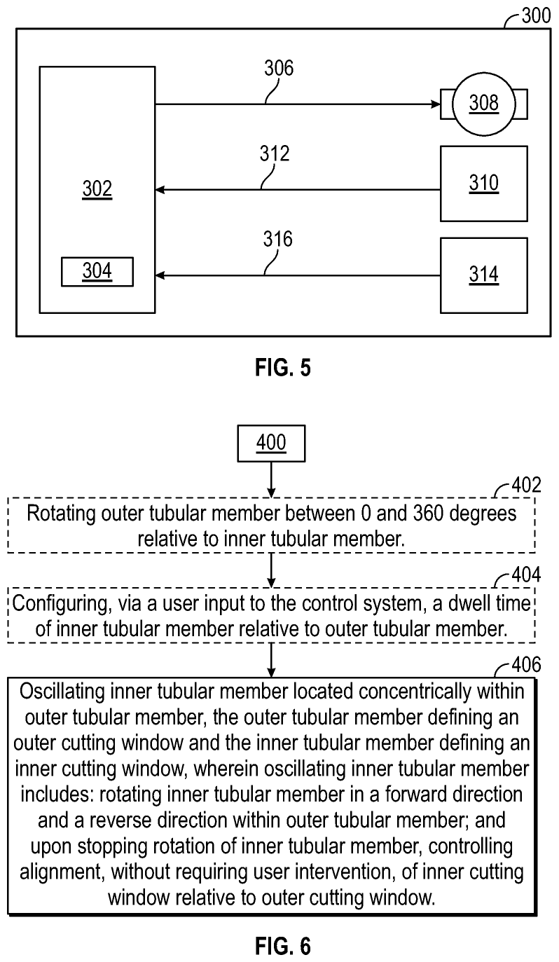

FIG. 5 illustrates an example of a schematic view of an angular positioning system 300 for a surgical cutting instrument. The angular positioning system 300 can be similar to the angular positioning system 102 discussed above with respect to, and shown in, FIGS. 1-2B. The angular positioning system 300 can include a controller 302, a user-interface 304, a motor 308, a first angular position sensor 310, and a second angular position sensor 314. The controller 302 can output or receive signals or data. For example, the controller 302 can be implemented in processing circuitry (e.g., hardwired or a processor), a programmable controller, such as a single or multi-board processor), a direct digital controller (DDC), a programmable logic controller (PLC), a system on a chip, a mobile device, a computer, or the like. The controller 302 include, or can be in communication, with a user-interface 304. For example, the user-interface 304 can be a touch-screen display or other electro-mechanical controls operable to relay control commands to the controller 302.

In the operation of some examples, a user can interact with the user-interface 304 to power on the controller 302. A user can configure one or more parameters or operations of the angular positioning system 300 by interacting with the user-interface 304. When the controller 302 is powered on, a user can activate the controller 302 to output a motor signal 306 to the motor 308. For example, the motor signal 306 can cause the motor 308 to rotate in a forward and in a reverse direction. In an example, the motor signal 306 can be configured by a user to, for example, cause the motor 308 to rotate at a specified speed, for specified length of time, or for a specified number of rotations.

In an example, the first angular position sensor 310 can output a first window signal 312 to the controller 302 based on an angular position of an inner hub, such as the inner hub 116 shown in FIGS. 1-2B. Alternatively, the first angular position sensor 310 can output the first window signal 312 to the controller 302 based on an angular position of a motor, such as the motor 130 shown in FIGS. 1-2B. The first window signal 312 can be a continuous signal or an intermittent signal, based on the type of sensing arrangement used. For example, the first angular position sensor 310 can intermittently output the first window signal 312 each time the motor completes a 360-degree rotation.

In an example, the angular positioning system 300 can include the second angular position sensor 314. The second angular position sensor 314 can output a second window signal 316 to the controller 302 based on an angular position of an outer hub 124, such as the outer hub 124 shown in FIGS. 1-2B. Alternatively, the second angular position sensor 314 can output the second window signal 316 to the controller 302 based on an angular position of an outer tubular member 110, such as the outer tubular member 110 shown in FIGS. 1-2B. The second window signal 316 can be a continuous signal or an intermittent signal, based on the type of sensing arrangement used. For example, the second angular position sensor 314 can intermittently output the second window signal 316 each time the angular position of the outer hub 124 is varied or changed, relative to the inner hub 116.

In an example, the controller 302 can receive the first window signal 312 from the first angular position sensor 310 to, using processing circuitry, control alignment of the inner cutting window 118 and the outer cutting window 126 by modifying the motor signal 306 in response to the first window signal 312, such as to bring the surgical cutting instrument 100 into the open position. In an example, the controller 302 can concurrently receive the first window signal 312 and the second window signal 316 from the first angular position sensor 310 and the second angular position sensor 314, respectively, to, using processing circuitry, control alignment of the inner cutting window 118 and the outer cutting window 126 by modifying the motor signal 306 in response to the first window signal 312 and the second window signal 316, such as to bring the surgical cutting instrument 100 into the open position. The controller 302 can thus, together with a surgical cutting instrument, such as the surgical cutting instrument shown and described in FIGS. 1-2B, be used to perform all, or a portion of, a surgical procedure on a patient.

FIG. 6 illustrates an example of a method of controlling a surgical cutting instrument. In this example, the method 400 includes operations such as optionally rotating an outer tubular member relative to an inner tubular member at 402, optionally configuring a dwell time of the inner tubular member relative to the outer tubular member at 404, and oscillating the inner tubular member within the outer tubular member at 406. In one or more examples, the method 400 can begin with an optional operation 402. Operation 402 can be rotating the outer tubular member between 0 and 360 degrees relative to the inner tubular member. For example, a user can manually rotate the outer tubular member between 0 and 360 degrees, relative to an inner tubular member and a housing of a surgical cutting instrument, in preparation for a surgical procedure.

In one or more examples, the method 400 can include an optional operation 404. Operation 404 can be configuring, via a user input to the control system, a dwell time of the inner tubular member relative to the outer tubular member. For example, a user can input to a controller, such as via a user interface in communication with the controller, a desired dwell time of the inner tubular member relative to the outer tubular member during an oscillation cycle, in preparation for a surgical procedure.

In one or more examples, the method 400 can include operation 406. Operation 406 can be oscillating an inner tubular member located concentrically within an outer tubular member, the outer tubular member defining an outer cutting window and the inner tubular member defining an inner cutting window, wherein oscillating the inner tubular member includes rotating the inner tubular member in a forward direction and a reverse direction within the outer tubular member; and upon stopping rotation of the inner tubular member, controlling alignment, without requiring user intervention, of the inner cutting window relative to the outer cutting window.

For example, the controller can receive a signal from a first angular position sensor corresponding to circumferential alignment between the inner cutting window member and the outer cutting window, or can concurrently process signals from first and second angular positions to determine when circumferential alignment between the inner cutting window and outer cutting window occurs. This can allow the controller to modify the motor signal to cause the motor to, concurrently, stop rotation of the motor, and bring the inner cutting window into alignment with the outer cutting window. In one or more examples, stopping the inner tubular member can include magnetically or optically monitoring an angular position of the inner tubular member relative to the outer tubular member with hall-effect, or optical sensors, respectively. For example, the first and/or the second angular position sensor can be a hall-effect or an optical sensor positioned within the housing of a surgical cutting instrument with respect to an inner hub and an outer hub.

In one or more examples, stopping the inner tubular member can include, after receiving a signal from the angular position sensor indicating circumferential alignment of the inner cutting window with the outer cutting window, stopping the inner tubular member within one or two subsequent 360 degree rotations of the inner tubular member. For example, the controller can detect circumferential alignment between the inner cutting window and the outer cutting windows and abruptly stop rotation of the motor to, concurrently, stop rotation of the motor, and algin the inner and outer cutting windows, such as by using active braking of the motor. Alternatively, the controller can detect circumferential alignment between the inner cutting window and the outer cutting windows, and based on a rotation speed of the motor, the controller can output a motor signal to rotate the inner tubular member a subsequent 360 degree rotation, at a reduced speed, to algin the inner and outer cutting windows.

The steps or operations of the method 400 are illustrated in a particular order for convenience and clarity. The discussed operations can be performed in parallel or in a different sequence without materially impacting other operations. The method 400 as discussed includes operations that can be performed by multiple different actors, devices, and/or systems. It is understood that subsets of the operations discussed in the method 400 can be attributable to a single actor device, or system, and could be considered a separate standalone process or method.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure.

This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### Notes and Examples

Example 1 is a powered surgical instrument such as comprising: a cutting assembly including: an outer tubular member defining an outer cutting window; an inner tubular member arranged to be rotatable concentrically within the outer tubular member and defining an inner cutting window; and a control system such as including: a controller; and an angular position sensor to provide to the controller, with respect to at least one of the inner and outer tubular members, an indication of angular orientation therebetween to allow the controller to control the angular orientation of the inner cutting window relative to the outer cutting window, without requiring user intervention, such that the inner and outer cutting windows are aligned when relative rotation between the inner and outer tubular members is stopped or paused.

In Example 2, the subject matter of Example 1 includes, wherein the relative rotation between the inner and outer tubular members is stopped or paused in response to a user input.

In Example 3, the subject matter of Examples 1-2 includes, wherein the control system is configured to receive the indication of angular orientation from the angular position sensor to offset the inner and outer cutting windows, without requiring user intervention, when relative rotation between the inner and outer tubular members is stopped or paused.

In Example 4, the subject matter of Examples 1-3 includes, wherein the control system is configured to receive the indication of angular orientation from the angular position sensor upon a first alignment of the inner and outer tubular members, and to stop or pause relative rotation between the inner and outer tubular members upon a second and subsequent alignment of the inner and outer tubular members.

In Example 5, the subject matter of Examples 1˜4 includes, wherein the control system is configured to stop or pause the relative rotation between the inner and outer tubular members without a user input.

In Example 6, the subject matter of Examples 4-5 includes, wherein the control system is configured to, based on a user input to the control system, control a dwell time for which the relative rotation between the inner and outer tubular members is paused.

In Example 7, the subject matter of Examples 1-6 includes, wherein the angular position sensor includes an optical encoder of a motor configured to be coupled to the inner tubular member and configured to rotate the inner tubular member in a forward direction and in a reverse direction.

Example 8 is a powered surgical instrument such as comprising: a housing; a cutting assembly such as including: an outer tubular member defining an outer cutting window extending through an annular surface of the outer tubular member; an outer hub coupled to a proximal portion of the outer tubular member; an inner tubular member arranged to be rotatable concentrically within the outer tubular member and defining an inner cutting window extending through an annular surface of the inner tubular member; an inner hub positioned within the housing and coupled to a proximal portion of the inner tubular member; and a control system configured to control rotation of the inner tubular member relative to the outer tubular member, the control system including or in communication with an angular position sensor for use in controlling alignment of the inner and outer cutting windows, without requiring user intervention, such that the inner and outer cutting windows are aligned when relative rotation between the inner and outer tubular members is stopped or paused.

In Example 9, the subject matter of Example 8 includes, a first angular position sensor to provide an indication of angular orientation of the inner tubular member to the control system; and a second angular position sensor to provide an indication of angular orientation of the outer tubular member to the control system, so that the inner and outer cutting windows are capable of being at least partially aligned by the control system, without requiring user intervention, when relative rotation between the inner and outer tubular members is stopped or paused; and wherein the first and the second angular position sensors each include an optical sensor located with respect to the inner hub and the outer hub, respectively.

In Example 10, the subject matter of Example 9 includes, wherein the first and the second angular position sensors each include a hall-effect sensor located with respect to the inner hub and the outer hub, respectively.

In Example 11, the subject matter of Examples 8-10 includes, wherein the control system is configured to, based on a user input, automatically control an angular position of the inner cutting window relative to the outer cutting window when the inner tubular member is stopped or paused between forward or reverse rotation of the inner tubular member.

In Example 12, the subject matter of Examples 8-11 includes, wherein the inner hub is coupled to the motor with a coupler configured to allow the cutting assembly to be detachable from the housing.

In Example 13, the subject matter of Examples 8-12 includes, wherein the control system is configured to, after receiving a signal from the angular position sensor indicating circumferential alignment of the inner cutting window with the outer cutting window, stop the inner tubular member within one or two subsequent 360 degree rotations of the inner tubular member.

Example 14 is a method for controlling a powered surgical instrument, the method such as comprising: oscillating an inner tubular member located concentrically within an outer tubular member, the outer tubular member defining an outer cutting window and the inner tubular member defining an inner cutting window, wherein oscillating the inner tubular member includes: rotating the inner tubular member in a forward direction and a reverse direction within the outer tubular member; and stopping rotation of the inner tubular member, wherein stopping rotation of the inner tubular member includes, controlling alignment, without requiring user intervention, of the inner cutting window relative to the outer cutting window.

In Example 15, the subject matter of Example 14 includes, wherein the method first comprises rotating the outer tubular member between 0 and 360 degrees relative to the inner tubular member.

In Example 16, the subject matter of Examples 14-15 includes, wherein the method first comprises rotating the outer tubular member 360 degrees relative to the inner tubular member.

In Example 17, the subject matter of Examples 14-16 includes, wherein the method first comprises configuring, via a user input to the control system, a dwell time of the inner tubular member relative to the outer tubular member.

In Example 18, the subject matter of Examples 14-17 includes, wherein stopping the inner tubular member includes magnetically monitoring an angular position of the inner tubular member relative to the outer tubular member using at least one hall-effect sensor.

In Example 19, the subject matter of Examples 14-18 includes, wherein controlling alignment, without requiring user intervention, of the inner cutting window relative to the outer cutting windows includes optically monitoring an angular position of the inner tubular member relative to the outer tubular member using at least one optical sensor.

In Example 20, the subject matter of Examples 14-19 includes, wherein controlling alignment, without requiring user intervention, of the inner cutting window relative to the outer cutting window includes, after receiving a signal from the angular position sensor indicating alignment of the inner cutting window with the outer cutting window, stopping the inner tubular member within one or two subsequent 360 degree rotations of the inner tubular member.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

What is claimed is:

1. A powered surgical instrument comprising:
a cutting assembly including:
an outer tubular member defining an outer cutting window;
an inner tubular member arranged to be rotatable concentrically within the outer tubular member and defining an inner cutting window; and
a control system including:
a controller; and
an angular position sensor to provide to the controller, with respect to at least one of the inner and outer tubular members, an indication of angular orientation therebetween to allow the controller to control the angular orientation of the inner cutting window relative to the outer cutting window, without requiring user intervention, such that the inner and outer cutting windows are aligned when relative rotation between the inner and outer tubular members is stopped or paused.

2. The surgical instrument of claim 1, wherein the relative rotation between the inner and outer tubular members is stopped or paused in response to a user input.

3. The surgical instrument of claim 1, wherein the control system is configured to receive the indication of angular orientation from the angular position sensor to offset the inner and outer cutting windows, without requiring user intervention, when relative rotation between the inner and outer tubular members is stopped or paused.

4. The surgical instrument of claim 1, wherein the control system is configured to receive the indication of angular orientation from the angular position sensor upon a first alignment of the inner and outer tubular members, and to stop or pause relative rotation between the inner and outer tubular members upon a second and subsequent alignment of the inner and outer tubular members.

5. The surgical instrument of claim 1, wherein the control system is configured to stop or pause the relative rotation between the inner and outer tubular members without a user input.

6. The surgical instrument of claim 4, wherein the control system is configured to, based on a user input to the control system, control a dwell time for which the relative rotation between the inner and outer tubular members is paused.

7. The surgical instrument of claim 1, wherein the angular position sensor includes an optical encoder of a motor configured to be coupled to the inner tubular member and configured to rotate the inner tubular member in a forward direction and in a reverse direction.

8. A powered surgical instrument comprising:
a housing;
a cutting assembly including:
an outer tubular member defining an outer cutting window extending through an annular surface of the outer tubular member;
an outer hub coupled to a proximal portion of the outer tubular member;
an inner tubular member arranged to be rotatable concentrically within the outer tubular member and defining an inner cutting window extending through an annular surface of the inner tubular member;
an inner hub positioned within the housing and coupled to a proximal portion of the inner tubular member; and
a control system configured to control rotation of the inner tubular member relative to the outer tubular member, the control system including or in communication with an angular position sensor for use in controlling alignment of the inner and outer cutting windows, without requiring user intervention, such that the inner and outer cutting windows are aligned when relative rotation between the inner and outer tubular members is stopped or paused.

9. The surgical instrument of claim 8, comprising:
a first angular position sensor to provide an indication of angular orientation of the inner tubular member to the control system; and
a second angular position sensor to provide an indication of angular orientation of the outer tubular member to the control system, so that the inner and outer cutting windows are capable of being at least partially aligned by the control system, without requiring user intervention, when relative rotation between the inner and outer tubular members is stopped or paused; and
wherein the first and the second angular position sensors each include an optical sensor located with respect to the inner hub and the outer hub, respectively.

10. The surgical instrument of claim 9, wherein the first and the second angular position sensors each include a hall-effect sensor located with respect to the inner hub and the outer hub, respectively.

11. The surgical instrument of claim 8, wherein the control system is configured to, based on a user input, automatically control an angular position of the inner cutting window relative to the outer cutting window when the inner tubular member is stopped or paused between forward or reverse rotation of the inner tubular member.

12. The surgical instrument of claim 8, wherein the inner hub is coupled to the motor with a coupler configured to allow the cutting assembly to be detachable from the housing.

13. The surgical instrument of claim 8, wherein the control system is configured to, after receiving a signal from the angular position sensor indicating circumferential alignment of the inner cutting window with the outer cutting window, stop the inner tubular member within one or two subsequent 360 degree rotations of the inner tubular member.

14. A method for controlling a powered surgical instrument, the method comprising:
oscillating an inner tubular member located concentrically within an outer tubular member, the outer tubular member defining an outer cutting window and the inner tubular member defining an inner cutting window, wherein oscillating the inner tubular member includes:
rotating the inner tubular member in a forward direction and a reverse direction within the outer tubular member; and
stopping rotation of the inner tubular member, wherein stopping rotation of the inner tubular member includes controlling alignment, without requiring user intervention, of the inner cutting window relative to the outer cutting window.

15. The method of claim 14, wherein the method first comprises rotating the outer tubular member between 0 and 360 degrees relative to the inner tubular member.

16. The method of claim 14, wherein the method first comprises rotating the outer tubular member 360 degrees relative to the inner tubular member.

17. The method of claim 14, wherein the method first comprises configuring, via a user input to the control system, a dwell time of the inner tubular member relative to the outer tubular member.

18. The method of claim 14, wherein stopping the inner tubular member includes magnetically monitoring an angular position of the inner tubular member relative to the outer tubular member using at least one hall-effect sensor.

19. The method of claim 14, wherein controlling alignment, without requiring user intervention, of the inner cutting window relative to the outer cutting window includes optically monitoring an angular position of the inner tubular member relative to the outer tubular member using at least one optical sensor.

20. The method of claim 14, wherein controlling alignment, without requiring user intervention, of the inner cutting window relative to the outer cutting window includes, after receiving a signal from the angular position sensor indicating alignment of the inner cutting window with the outer cutting window, stopping the inner tubular member within one or two subsequent 360 degree rotations of the inner tubular member.
